(19) United States
(12) Patent Application Publication
(10) Pub. No.: US 2025/0136561 A1
(43) Pub. Date: May 1, 2025

(54) COMPOSITIONS OF MATTER FROM UNSATURATED NITRILES

(71) Applicant: INV NYLON CHEMICALS AMERICAS, LLC, Wilmington, DE (US)

(72) Inventors: Stewart Forsyth, Wichita, KS (US); Isaac K. Iverson, Wichita, KS (US); Ameena Mitha, Bridge City, TX (US); Eric Robert Sirianni, Bridge City, TX (US)

(21) Appl. No.: 18/690,754

(22) PCT Filed: Sep. 21, 2021
(86) PCT No.: PCT/IB2022/058888

§ 371 Date: Mar. 11, 2024

Related U.S. Application Data
(60) Provisional application No. 63/246,565, filed on Sep. 21, 2021.

(51) Int. Cl. C07D 251/18; C07C 213/00; C07C 215/08; C07C 215/20; C07C 215/28; C07C 227/26; C07C 229/12; C07C 229/14; C07C 231/06; C07C 237/06; C07C 237/14; C07C 237/20

(52) U.S. Cl. CPC C07D 251/18; C07C 213/00; C07C 215/08; C07C 215/20; C07C 215/28; C07C 227/26; C07C 229/12; C07C 229/14; C07C 231/06; C07C 237/06; C07C 237/14; C07C 237/20; C07C 2601/14

(57) ABSTRACT

A new composition of matter has the molecular structure [I]: Formula (I) wherein R₁ is an alkyl, cycloalkyl, aralkyl group having ≥1 to ≤10 carbon atoms; R₂ is selected from the group consisting of an aliphatic C₁-C₁₀ group, an unsaturated C₁-C₁₀ chain, C₄-C₉ group with a cyclic portion and C₄-C₁₀ group with an aromatic portion, and combinations thereof; R₃ is selected from the group consisting of hydrogen, an aliphatic C₁-C₁₀ group, an unsaturated C₂-C₁₀ chain, a C₄-C₁₀ group with a cyclic portion, C₄-C₁₀ group with an aromatic portion, and combinations thereof; and R₄ is a terminal functional group selected from the group consisting of amide [—CONH₂], acid [—COOH], amine [—CH₂—NH₂], guanamine [—(C₃N₃)—(NH₂)₂], organic heterocyclic [—CHN₄], and combinations thereof; and wherein said composition of matter of the molecular structure [I] excludes 2-[(3-amino-1-ethylpropyl)amino]ethanol.

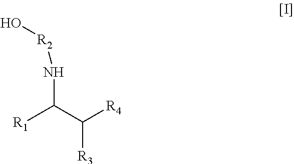

### FIELD

This present application relates to new compositions of matter derived from unsaturated organo-nitriles.

### BACKGROUND

Certain unsaturated organo-nitriles, such as 3-pentenenitrile and methyl-butenenitriles, are the by-products of important commercial processes, such as the production of adiponitrile by hydrocyanation of butadiene, and there is interest in developing new uses for these materials.

According to embodiments of the present disclosure, it has now been found that unsaturated organo-nitriles can be converted in a simple two stage process to novel multi-functional compounds useful in a variety of applications, such as crossing linking agents, particularly in the formation of polyamides, as well as chelating agents, curatives and in coating systems.

### SUMMARY

In one aspect, the present application is directed to a new composition of matter having the molecular structure [I]:

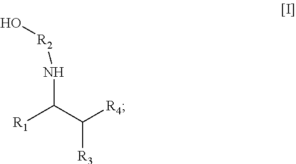

wherein,
R₁ is an alkyl, cycloalkyl, or aralkyl group having ≥1 to ≤10 carbon atoms which optionally comprises unsaturated fractions;
R₂—is selected from the group consisting of an aliphatic C₁-C₁₀ group, an unsaturated C₂-C₁₀ chain, a C₄-C₉ group with a cyclic portion, a C₄-C₁₀ group with an aromatic portion, and combinations thereof;
R₃ is selected from the group consisting of hydrogen, an aliphatic C₁-C₁₀ chain, an unsaturated C₂-C₁₀ chain, a C₄-C₁₀ group with a cyclic portion and a C₄-C₁₀ group with an aromatic portion, and combinations thereof; and
R₄ is a terminal functional group selected from the group consisting of amide [—CONH₂], acid [—COOH], amine [—CH₂—NH₂], guanamine [—(C₃N₃)—(NH₂)₂], organic heterocyclic [—CHN₄], and combinations thereof;
and wherein, said composition of matter having the molecular structure [I] excludes 2-[(3-amino-1-ethylpropyl)amino]ethanol.

In a further aspect, the present application is directed to a method of producing a composition of matter having the molecular structure [I]:

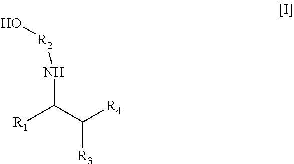

the method comprising;
(a) feeding an unsaturated organonitrile, a hydroxy-amine, and optionally, a solvent to a first reaction zone,
(b) maintaining the temperature and pressure in the first reaction zone for time sufficient to obtain a hydroxy-aminonitrile of the molecular structure [II]:

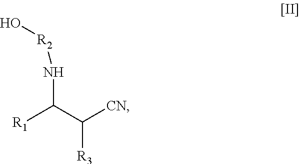

(c) feeding the hydroxy-aminonitrile obtained from step b) to a second reaction zone, and
(d) maintaining the reaction conditions in the second reaction zone for time sufficient for functionalizing the hydroxy-aminonitrile of the molecular structure [II] to obtain a composition of matter of the molecular structure [I]:
   wherein,
R₁ is an alkyl, cycloalkyl, or aralkyl group having ≥1 to ≤10 carbon atoms which optionally comprises unsaturated fractions;
R₂—is selected from the group consisting of an aliphatic C₁-C₁₀ group, an unsaturated C₂-C₁₀ chain, a C₄-C₉ group with a cyclic portion, a C₄-C₁₀ group with an aromatic portion, and combinations thereof;
R₃ is selected from the group consisting of hydrogen, an aliphatic C₁-C₁₀ chain, an unsaturated C₂-C₁₀ chain, a C₄-C₁₀ group with a cyclic portion and a C₄-C₁₀ group with an aromatic portion, and combinations thereof; and
R₄ is a terminal functional group selected from the group consisting of amide [—CONH₂], acid [—COOH], amine [—CH₂—NH₂], guanamine [—(C₃N₃)—(NH₂)₂], organic heterocyclic [—CHN₄], and combinations thereof.

### DETAILED DESCRIPTION

Disclosed herein is a new composition of matter of the molecular structure [I], as described below:

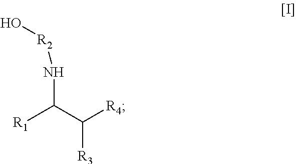

wherein
R₁ is an alkyl, cycloalkyl, aralkyl group having ≥1 to ≤10 carbon atoms which optionally comprises unsaturated fractions;
R₂ is selected from the group consisting of an aliphatic C₁-C₁₀ group, an unsaturated C₂-C₁₀ chain, a C₄-C₉ group with a cyclic portion and a C₄-C₁₀ group with an aromatic portion;
R₃ is selected from the group consisting of hydrogen, an aliphatic C₁-C₁₀ chain, an unsaturated C₂-C₁₀ chain, a C₄-C₁₀ group with a cyclic portion and a C₄-C₁₀ group with an aromatic portion; and
R₄ is a terminal functional group selected from the group consisting of amide [—CONH₂], acid [—COOH], amine [—CH₂—NH₂], guanamine [—(C₃N₃)—(NH₂)₂] and organic heterocyclic [—CHN₄];
   and wherein, said composition of matter having the molecular structure [I] excludes 2-[(3-amino-1-ethylpropyl)amino]ethanol, which is a known material having CAS No. 2294600-31-8.

The new compositions of matter of the molecular structure [I] are useful as crossing linking agents, particularly in the formation of polyamides, as well as chelating agents, curatives and in coating systems.

Also disclosed herein is a method of producing a composition of matter of the molecular structure [I]:

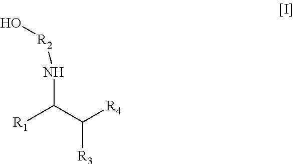

the method comprising;
(a) feeding an unsaturated organonitrile, a hydroxy-amine, and optionally, a solvent to a first reaction zone,
(b) maintaining the temperature and pressure in the first reaction zone for time sufficient to obtain a hydroxy-aminonitrile of the molecular structure [II]:

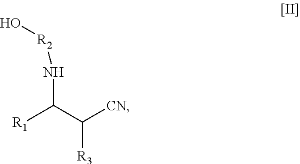

(c) feeding the hydroxy-aminonitrile obtained from step b) to a second reaction zone, and
(d) maintaining the reaction conditions in the second reaction zone for time sufficient for functionalizing the hydroxy-aminonitrile of the molecular structure [II] to obtain a composition of matter of the molecular structure [I]:
   wherein,
R₁ is an alkyl, cycloalkyl, or aralkyl group having ≥1 to ≤10 carbon atoms which optionally comprises unsaturated fractions;
R₂—is selected from the group consisting of an aliphatic C₁-C₁₀ group, an unsaturated C₂-C₁₀ chain, a C₄-C₉ group with a cyclic portion, a C₄-C₁₀ group with an aromatic portion, and combinations thereof;
R₃ is selected from the group consisting of hydrogen, an aliphatic C₁-C₁₀ chain, an unsaturated C₂-C₁₀ chain, a C₄-C₁₀ group with a cyclic portion and a C₄-C₁₀ group with an aromatic portion, and combinations thereof; and
R₄ is a terminal functional group selected from the group consisting of amide [—CONH₂], acid [—COOH], amine [—CH₂—NH₂], guanamine [—(C₃N₃)—(NH₂)₂], organic heterocyclic [—CHN₄], and combinations thereof.

In some embodiments, R₁ in the composition of matter of molecular structure [I] can be an alkyl group selected from the group consisting of methyl [—CH₃]; ethyl [—C₂H₅]; propyl [—C₃H₇], butyl [—C₄H₉], pentyl [—C₅H₁₁], hexyl [—C₆H₁₃], heptyl [—C₇H₁₅], octyl [—C₈H₁₇], nonyl [—C₉H₁₉] and decyl [—C₁₀H₂₁].

In some embodiments, R₂ in the composition of matter of molecular structure [I] may be a —[CH₂]ₘ— aliphatic group with the numerical value of “m” from 1 to 10. Specifically, R₂ may be selected from the group consisting of methylene [—CH₂—], ethylene [—C₂H₄—], propylene [C₃H₆—], butylene [—C₄H₈—], pentylene [—C₅H₁₀—], hexylene [—C₆H₁₂—], heptylene [—C₇H₁₄—], octylene [—C₈H₁₆—], nonylene [—C₉H₁₈—] and decylene [—C₁₀H₂₀—].

In other embodiments, R₂ in the composition of matter of molecular structure [I] may be a C₂-C₁₀ chain including unsaturation, for example, the presence of one or more double bonds [—C═C—], triple bonds [—C≡C—], or both. Non-limiting examples include [—CH═CH—], [—CH2—CH═CH—CH2—], [—CH2—CH2—C≡C—CH2—], and combinations thereof.

In other embodiments, R₂ in the composition of matter of molecular structure [I] may be a C₄-C₉ group with a cyclic portion selected from the group consisting of cyclo-butyl [—CH—(CH₂)₂—CH—], cyclo-pentyl [—CH—(CH₂)₃—CH—], cyclo-hexyl [—CH—(CH₂)₄—CH—], cyclo-heptyl [—CH—(CH₂)₅—CH—], cyclo-octyl [—CH—(CH₂)₆—CH—], cyclo-nonyl [—CH—(CH₂)₇—CH—] portions.

In some embodiments, R₃ in the composition of matter of molecular structure [I] may be selected from the group consisting of methyl [—CH₃]; ethyl [—C₂H₅]; propyl [—C₃H₇], butyl [—C₄H₉], pentyl [—C₅H₁₁], hexyl [—C₆H₁₃], heptyl [—C₇H₁₅], octyl [—C₈H₁₇], nonyl [—C₉H₁₉] and decyl [—C₁₀H₂₁].

In other embodiments, R₃ in the composition of matter of molecular structure [I] may be a C₂-C₁₀ chain including unsaturation, for example, the presence of one or more double bonds [—C═C—], triple bonds [—C≡C—], or both. Non-limiting examples include [—CH═CH—], [—CH2—CH═CH—CH2—], [—CH2—CH2—C≡C—CH2—], and combinations thereof.

In other embodiments, R₃ in the composition of matter of molecular structure [I] may be a C₄-C₉ group with a cyclic portion selected from the group consisting of cyclo-butyl [—CH—(CH₂)₂—CH—], cyclo-pentyl [—CH—(CH₂)₃—CH—], cyclo-hexyl [—CH—(CH₂)₄—CH—], cyclo-heptyl [—CH—(CH₂)₅—CH—], cyclo-octyl [—CH—(CH₂)₆—CH—], cyclo-nonyl [—CH—(CH₂)₇—CH—] portions.

The disclosed composition of matter of the molecular structure [I] can be prepared from an intermediate compound of the molecular structure [II], as described below:

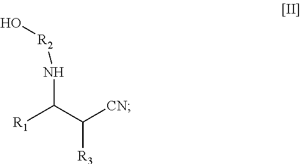

wherein R₁, R₂ and R₃ are as described above.

The intermediate compound of the molecular structure [II] may be obtained by the reaction between a candidate unsaturated organonitrile and a hydroxy-amine, as shown in Reaction 1 scheme below:

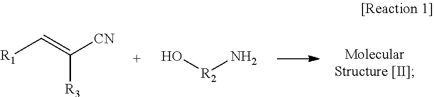

wherein R₁, R₂ and R₃ are as described above.

In some embodiments, the unsaturated organonitrile employed in Reaction I can have at least one unsaturation on its backbone within three carbon atom locations numbered from its nitrile end.

In some embodiments, the unsaturated organonitrile employed in Reaction I can comprise a five-carbon unsaturated nitrile. For example, the five-carbon unsaturated nitrile can be selected from the group consisting of 2-pentenenitrile [2PN], 3-pentenenitrile [3PN], 4-pentenenitrile [4PN], 2-methyl-2-butenenitrile [2M2BN], 2-methyl-3-butenenitrile [2M3BN] and cis/trans mixtures.

In some embodiments, the hydroxy-amine employed in Reaction I is selected from the group consisting of a C₁-C₁₀ aliphatic alkanolamine, a C₂-C₁₀ unsaturated alkanolamine, a C₄-C₁₀ cyclic alkanolamine, a C₄-C₁₀ aromatic alkanolamine and mixtures thereof. Suitable C₁-C₁₀ aliphatic alkanolamines include methanol amine, ethanol amine, butanol amine, pentanol amine, hexanol amine, heptanol amine, octanol amine, nonanol amine, decanol amine and isomers and mixtures thereof. In some embodiments, the C₁-C₁₀ unsaturated alkanolamine can be chosen from the group consisting of methanol amine, ethanol amine and isomers and mixtures thereof. Suitable cyclic alkanolamines include amino-cyclobutanol, amino-cyclopentanol, amino-cyclohexanol, amino-cycloheptanol, amino-cyclooctanol, amino-cyclononanol, amino-cyclodecanol and isomers and mixtures thereof. In some embodiments, the cyclic alkanolamine can be chosen from the group consisting of 2-amino-cyclobutanol, 2-amino-cyclohexanol, 3-amino-cyclohexanol, 4-amino-cyclohexanol and isomers and mixtures thereof. Suitable C₄-C₁₀ aromatic alkanolamines include amino-phenols, namely, 2-aminophenol, 3-aminophenol and 4-aminophenol, as well as 4-hydroxy-diphenyleneamine.

A variety of solvents can be used in conjunction with Reaction I, examples of which include water, alcohols, alkanes, ethers and esters.

In some embodiments, Reaction 1 can be conducted at a temperature of 0 to 100° C., preferably 20 to 75° C. and a pressure sufficient to maintain the reactants in the liquid phase for a time from 4 to 72 hours.

The intermediate compound of molecular structure [II] produced by reaction I may be referred to as hydroxy-aminonitrile, or alternatively as aminonitrile alcohol.

The disclosed composition of matter of the molecular structure [I] may be obtained by converting the intermediate compound [II] to yield the desired “—R₄” functionality, as shown in Reaction 2 scheme below:

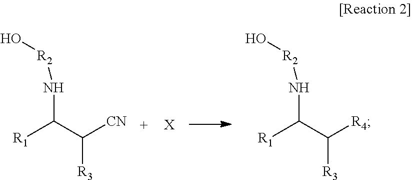

wherein R₁, R₂, R₃ and R₄ are as described above.

In Reaction 2 scheme above, the term “X” represents a chemical agent or agents that participate(s) in converting the cyanide [—CN] group present in the intermediate of the molecular structure [II] to the desired “—R₄” functionality. The chemical agent(s) “X” may include water, hydrogen, CO, CO₂, ammonia, syn gas, amines, cyano-amides, imines, acids, bases, azide salts, and including suitable catalysts to promote the reaction.

In one embodiment, X can include water that hydrolytically reduces the intermediate of the molecular structure [II] to convert cyanide to an amide [—CN→—CONH₂] functionality for “—R₄” present in the composition of the molecular structure [I]. Such a reaction can be conducted at a temperature of 0 to 100° C., preferably 20 to 75° C., and a pressure sufficient to maintain the reactants in the liquid phase for a time from 4 to 72 hours.

In another embodiment, X can include water and a base catalyst, such as sodium hydroxide, that hydrolyzes the intermediate of the molecular structure [II] to convert cyanide to an acid [—CN→—COOH] functionality for “—R₄” present in the composition of the molecular structure [I]. Such a reaction can be conducted at a temperature of 50 to 100° C. and a pressure of 100 to 1000 psig for a time from 4 to 72 hours.

In yet another embodiment, X can be a hydrogen- and ammonia-containing medium that reductively aminates the intermediate of the molecular structure [II] convert cyanide to an amine [—CN→—CH₂—NH₂] functionality for “—R₄” present in the composition of the molecular structure [I]. Such a reaction can be conducted at a temperature of 0 to 100° C., preferably 20 to 75° C., and a pressure sufficient to maintain the reactants in the liquid phase for a time from 4 to 72 hours.

In another embodiment, X can be a dicyandiamide that reacts with the intermediate of the molecular structure [II] convert cyanide to a guanamine [—CN→—(C₃N₃)—(NH₂)₂] functionality for “—R₄” present in the composition of the molecular structure [I]. Such a reaction can be conducted at a temperature of 0 to 100° C., preferably 20 to 75° C., and a pressure sufficient to maintain the reactants in the liquid phase for a time from 4 to 72 hours.

In one other embodiment, X can include an azide salt and catalyst that reacts with the intermediate of the molecular structure [II] convert cyanide to an organic heterocyclic [—CN→—CHN₄] functionality for “—R₄” present in the composition of the molecular structure [I]. Sodium azide salts and iodine catalyst may be used. Such a reaction can be conducted at a temperature of 0 to 100° C., preferably 20 to 75° C., and a pressure sufficient to maintain the reactants in the liquid phase for a time from 4 to 72 hours.

### EXAMPLES

Various embodiments of the present disclosure can be better understood by reference to the following Examples which are offered by way of illustration. The present disclosure is not limited to the Examples given herein.

### Example 1

A hydroxy-aminonitrile, namely, 3-[(2-hydroxyethyl)amino]pentanenitrile [C₇H₁₄N₂O; CAS #1155164-96-7] was prepared from 3-pentenenitrile (2PN) and mono-ethanol amine in accordance with Reaction 1 scheme. To a 40 mL glass vial equipped with a magnetic stir bar was added 2-aminoethanol (0.9 g, 15 mmol, 2.5 equiv), 3-pentenenitrile (0.5 g, 6.0 mmol, 1.0 equiv) and water (37 wt %, 2.4 mL). The reaction vial was kept under nitrogen, sealed and heated to 90° C. for 48 hours with vigorous stirring on a heating block resulting in 95% consumption of the nitrile starting material based on 1H NMR analysis. After cooling to room temperature, the volatiles were removed under reduced pressure on a rotary evaporator to give the desired product (0.85 g, 99% yield, ˜80% purity) as a yellow oil. 1H NMR (400 MHz, CDCl3) δ=3.64 (t, 2H), 2.81 (m, 3H), 2.50 (dq, 2H), 0.98 (t, 3H) ppm.

Partial hydrolysis of the resultant hydroxy-aminonitrile yielded the hydroxy-amino amide shown in Table 1.

### Example 2

To a 40 mL glass vial equipped with a magnetic stir bar was added 4-aminophenol (2.3 g, 21 mmol, 1.0 equiv), 3-pentenenitrile (4.2 g, 52 mmol, 2.5 equiv) and water (11.0 mL, 37 wt %). The reaction vial was kept under nitrogen, sealed, and heated to 90° C. with vigorous stirring on a heating block. After 24 hours, LCMS analysis indicated that there was no conversion to the desired product and the starting materials remained. After 24 hours, sodium hydroxide (0.4 g, 10.3 mmol, 0.5 equiv) was added to the reaction mixture and a large exotherm was observed. The reaction mixture was heated and stirred for an additional 4 days, at which time LC/MS analysis indicated ˜35% of the desired target product, 3-([4-hydroxyphenyl]amino)pentanenitrile.

### Example 3

A solution of crude 3-((2-hydroxyethyl)amino)pentanenitrile (0.32 g, 2.3 mmol, 1 equiv) and 6N aqueous sodium hydroxide (1.1 mL, 6.8 mmol 3.0 equiv) was stirred at room temperature for 24 hours. The reaction progressed slowly and was heated at 90° C. for an additional 24 hours to provide the corresponding sodium salt of the hydroxy-amino acid in an aqueous solution. The solution was neutralized and analyzed. Crude 1H-NMR indicated the starting material was fully consumed, and the presence of the desired product. However, impurities from pentenenitriles were also observed.

### Example 4

A solution of crude 3-((2-hydroxyethyl)amino)pentanenitrile (2.8 g, 19.7 mmol, 1 equiv), dicyandiamide (1.2 equiv) and potassium hydroxide (0.24 g, 4.3 mmol, 0.2 equiv, 5 wt %) in 1-methoxy-2-propanol (50 mL) was refluxed for 7 hours under nitrogen resulting in 22% conversion to hydroxyamino-guanamine along with several other peaks as determined by LCMS analysis.

### Example 5

A mixture of crude 3-((2-hydroxyethyl)amino)pentanenitrile (5.0 g, 1 equiv) and Raney nickel (2800) (wet, 1.0 equiv) in tetrahydrofuran (2 vol) was hydrogenated @250 psi at 90° C. with overhead stirring for 16 hours. Once the starting material was consumed the reaction mixture was cooled to room temperature and filtered through celite, which was washed with methanol (3×50 mL). The filtrate was concentrated in vacuo to provide crude hydroxy-amino amine as a brown oil (3.67 g, 71% yield).

Table 1 below summarizes Examples 1 to 5.

TABLE 1

| | | Intermediate | |
|---|---|---|---|
| Ex. | | Compound [II]- | Composition of Matter of |
| ID. | Unsaturated Nitrile | [Hydroxy-Aminonitrile] | Molecular Structure [I] |
| 1 | 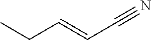 | 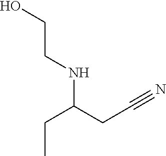 | 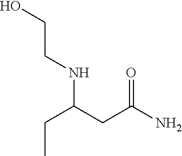 |
| | 2-Pentenenitrile | 3-[(2- | Hydroxy-amino amide |
| | | hydroxyethyl)amino] | |
| | | Pentanenitrile | |
| | | CAS No. 1155164-96-7 | |
| 2 | As Example 1 | 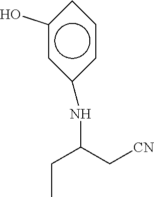 | |
| 3 | As Example 1 | As Example 1 | 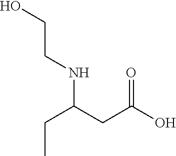 |
| | | | Hydroxy-amino acid |
| 4 | As Example 1 | As Example 1 | 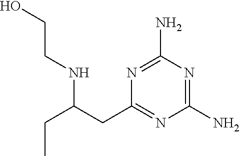 [SMILES: CCC(Cc1nc(N)nc(N)n1)NCCO] |
| | | | Hydroxy-amino guanamine |
| 5 | As Example 1 | As Example 1 | 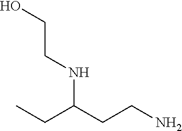 |
| | | | 2-[(3-amino-1- |
| | | | ethylpropyl)amino] Ethanol |
| | | | CAS No. 2294600-31-8 |

In another embodiment, the hydroxy-aminonitrile of Example 1 is hydrolytically reduced [per Reaction 2 scheme] to obtain the new composition of matter, a hydroxy-amino amide of the structure:

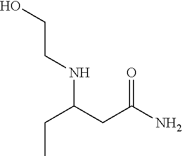

In further embodiment, the hydroxy-aminonitrile of Example 1 undergoes reaction with an azide salt and a catalyst to obtain the new composition of matter, a hydroxy-amino tetrazol of the structure [I]:

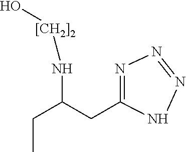

### Examples 6-9

As shown in Table 2, the Reaction 1 scheme for the reaction of 2-PN with mono-butanol amine or 4-aminobutanol [HO—[CH₂]₄—NH₂] yields the corresponding hydroxy-aminonitrile shown in the second column of Table 2.

The intermediate formed from 2-PN and mono-butanol amine and shown in Table 2, second column is described as the molecular structure [II], wherein “—R₁” is —CH₂—CH₃; “—R₂—” is —[CH₂]₄—; and “—R₃” is —H₂.

On functionalization as summarized in the third column of Table 2, the new compositions of matter shown in the fourth column of Table 2 are produced.

TABLE 2

| | Intermediate | | |
|---|---|---|---|
| Ex. | Compound [II]- | | Composition Matter of |
| ID. | [Hydroxy-Aminonitrile] | Via Reaction 2 Scheme | Molecular Structure [I] |
| 6 | 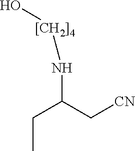 | which upon reductive hydrogenation in the presence of ammonia and hydrogen, yields | 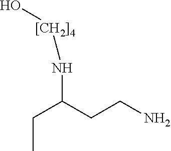 |
| 7 | As Example 6 | which upon hydrolysis, yields | 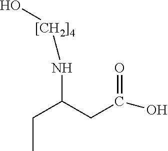 |
| 8 | As Example 6 | which upon hydrolytic reduction, yields | 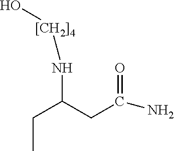 |
| 9 | As Example 6 | which upon reaction with dicyandiamide, yields | 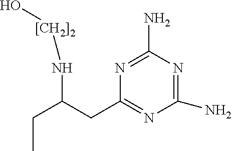 |

### Examples 10-13

As shown in Table 3, the Reaction 1 scheme for reaction of 2-PN with amino-cyclohexanol [HO—[C₆H₁₀]—NH₂] yields a hydroxy-aminonitrile as shown in the second column of Table 3.

The intermediate formed from 2-PN and amino-cyclohexanol and shown in Table 3, second column is described as the molecular structure [II], wherein “—R₁” is —CH₂—CH₃; “—R₂—” is —[C₆H₁₀]—; and “—R₃” is —H₂.

On functionalization as summarized in the third column of Table 3, the new compositions of matter shown in the fourth column of Table 3 are produced.

TABLE 3

| | Intermediate | | |
|---|---|---|---|
| | Compound [II]- | | Composition Matter of |
| Ex. ID. | [Hydroxy-Aminonitrile] | Via Reaction 2 Scheme | Molecular Structure [I] |
| 10 | 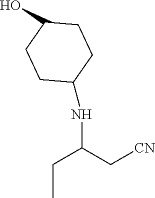 | which upon reductive hydrogenation in the presence of ammonia and hydrogen, yields | 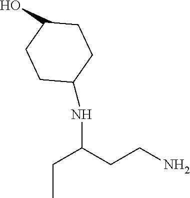 |
| 11 | As Example 10 | upon hydrolysis, yields | 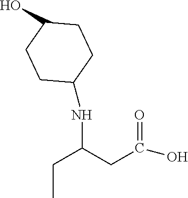 |
| 12 | As Example 10 | upon hydrolytic reduction, yields | 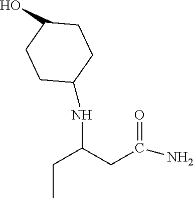 |
| 13 | As Example 10 | which upon reaction with dicyandiamide, yields | 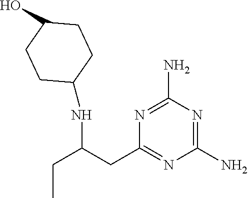 |

### Examples 14-17

As shown in Table 4, the Reaction 1 scheme for the reaction of 2-PN with 3-aminophenol [HO—[C₆H₄]—NH₂] yields a hydroxy-aminonitrile as shown in the second column of Table 4.

The intermediate formed from 2-PN and 3-aminophenol and shown in Table 4, second column is described as the molecular structure [II], wherein “—R₁” is —CH₂—CH₃; “—R₂—” is —[C₆H₄]—; and “—R₃” is —H₂.

On functionalization as summarized in the third column of Table 4, the new compositions of matter shown in the fourth column of Table 4 are produced.

TABLE 4

| | Intermediate | | |
|---|---|---|---|
| | Compound [II]- | | Composition Matter of |
| Ex. ID. | [Hydroxy-Aminonitrile] | Via Reaction 2 Scheme | Molecular Structure [I] |
| 14 | 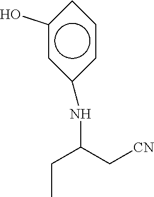 | which upon reductive hydrogenation in the presence of ammonia and hydrogen, yields | 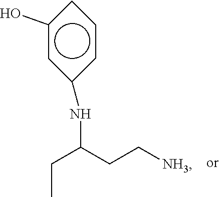 |
| 15 | ″ | upon hydrolysis, yields | 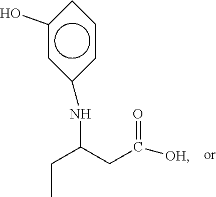 |
| 16 | ″ | upon hydrolytic reduction, yields | 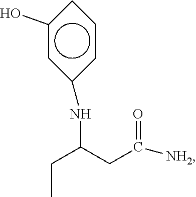 |
| 17 | ″ | which upon reaction with dicyandiamide, yields | 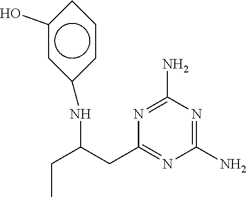 |

### Examples 18-21

Table 5 provides additional illustrative examples of the composition of matter of the molecular structure [I], obtained according to Reaction 1 followed by Reaction 2 schemes. In Table 5, the non-limiting examples of the starting candidate unsaturated branched nitriles are 2-methyl-2-butenenitrile or “2M2BN” [cis/trans] and 2-methyl-3-butenenitrile or “2M3BN” [cis/trans]. Isomerization of 2M2BN to 2M3BN or reverse in the presence of a base medium is known. Examples in Table 5 are obtained by using mono-ethanolamine in Reaction 1 scheme.

The intermediate formed from either 2M2BN or 2M3BN and mono-ethanolamine and shown in Table 5, third column is described as the molecular structure [II], wherein “—R₁” is —CH₃; “—R₂—” is —[CH₂]₂—; and “—R₃” is —CH₃.

On functionalization, the new compositions of matter shown in the fourth column of Table 5 are produced.

TABLE 5

| | Candidate Unsaturated | Intermediate Compound [II]- | New Composition of Matter of |
|---|---|---|---|
| Ex. ID | Nitrile | [Hydroxy-Amino Nitrile] | Molecular Structure [I] |
| 18 | 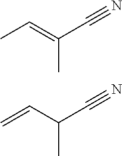 | 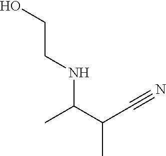 | 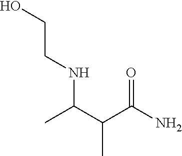 |
| | 2M2BN, 2M3BN | 3-[(2-hydroxyethyl)amino]- | Hydroxy-amino amide |
| | | 2-methyl-Butanenitrile | |
| | | CAS No. 1849323-94-9 | |
| 19 | As Example 18 | As Example 18 | 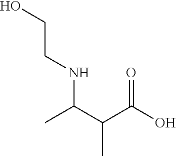 |
| | | | Hydroxy-amino acid |
| 20 | As Example 18 | As Example 18 | 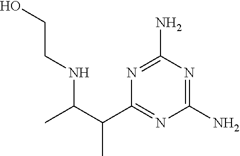 [SMILES: CC(NCCO)C(C)c1nc(N)nc(N)n1] |
| | | | Hydroxy-amino guanamine |
| 21 | As Example 18 | As Example 18 | 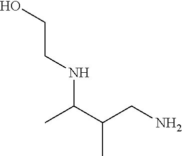 |
| | | | Hydroxy-amino amine |

Tables 1-5 give various illustrative examples which are not limiting to only those shown. Persons skilled in the art will very well appreciate other variations of the new compositions of matter of the molecular structure [I] that can be obtained via the formation of corresponding intermediate compounds [II].

Also, it is to be understood that pure, crude as well as mixtures of nitriles may be used in preparing the intermediate compounds [II] and followed by their conversion to the corresponding compositions of matter of the molecular structure [I].

1. A composition of matter of the molecular structure [I]: 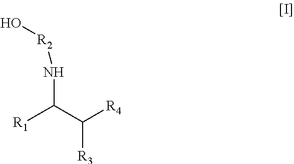
wherein,
R₁ is an alkyl, cycloalkyl, aralkyl group having ≥1 to ≤10 carbon atoms which optionally comprises unsaturated fractions;
R₂ is selected from the group consisting of an aliphatic C₁-C₁₀ group, an unsaturated C₂-C₁₀ chain, a C₄-C₉ group with a cyclic portion, a C₄-C₁₀ group with an aromatic portion, and combinations thereof;
R₃ is selected from the group consisting of hydrogen, an aliphatic C₁-C₁₀ chain, an unsaturated C₂-C₁₀ chain, a C₄-C₁₀ group with a cyclic portion, C₄-C₁₀ group with an aromatic portion, and combinations thereof, and
R₄ is a terminal functional group selected from the group consisting of amide [—CONH₂], acid [—COOH], amine [—CH₂—NH₂], guanamine [—(C₃N₃)—(NH₂)₂], organic heterocyclic [—CHN₄], and combinations thereof;
and wherein, said composition of matter of the molecular structure [I] excludes 2-[(3-amino-1-ethylpropyl)amino]ethanol.

2. The composition of matter of claim 1, wherein R₁ is selected from the group consisting of methyl [—CH₃]; ethyl [—C₂H₅]; propyl [—C₃H₇], butyl [—C₄H₉], pentyl [—C₅H₁₁], hexyl [—C₆H₁₃], heptyl [—C₇H₁₅], octyl [—C₈H₁₇], nonyl [—C₉H₁₉] and decyl [—C₁₀H₂₁].

3. The composition of matter of claim 1, wherein R₂ is selected from the group consisting of methylene [—CH₂—], ethylene [—C₂H₄—], propylene [C₃H₆—], butylene [—C₄H₈—], pentylene [—C₅H₁₀—], hexylene [—C₆H₁₂—], heptylene [—C₇H₁₄—], octylene [—C₈H₁₆—], nonylene [—C₉H₁₈—] and decylene [—C₁₀H₂₀—].

4. The composition of matter of claim 1, wherein R₂ comprises an unsaturated group selected from [—CH═CH—], [—CH₂—CH═CH—CH₂—], [—CH₂—CH₂—C≡C—CH₂—] and combinations thereof.

5. The composition of matter of claim 1, wherein R₂ comprises a cyclic portion selected from the group consisting of cyclo-butyl [—CH—(CH₂)₂—CH—], cyclo-pentyl [—CH—(CH₂)₃—CH—], cyclo-hexyl [—CH—(CH₂)₄—CH—], cyclo-heptyl [—CH—(CH₂)₅—CH—], cyclo-octyl [—CH—(CH₂)₆—CH—], cyclo-nonyl [—CH—(CH₂)₇—CH—] and combinations thereof.

6. The composition of matter of claim 1, wherein R₃ is hydrogen.

7. The composition of matter of claim 1, wherein R₃ is aliphatic C₁-C₁₀ chain selected from the group consisting of methyl [—CH₃]; ethyl [—C₂H₅]; propyl [—C₃H₇], butyl [—C₄H₉], pentyl [—C₅H₁₁], hexyl [—C₆H₁₃], heptyl [—C₇H₁₅], octyl [—C₈H₁₇], nonyl [—C₉H₁₉] and decyl [—C₁₀H₂₁].

8. The composition of matter of claim 1, wherein R₃ comprises an unsaturated group selected from [—CH═CH—], [—CH₂—CH═CH—CH₂—], [—CH₂—CH₂—C≡C—CH₂—] and combinations thereof.

9. The composition of matter of claim 1, wherein R₃ comprises a cyclic portion selected from the group consisting of cyclo-butyl [—CH—(CH₂)₂—CH—], cyclo-pentyl [—CH—(CH₂)₃—CH—], cyclo-hexyl [—CH—(CH₂)₄—CH—], cyclo-heptyl [—CH—(CH₂)₅—CH—], cyclo-octyl [—CH—(CH₂)₆—CH—], cyclo-nonyl [—CH—(CH₂)₇—CH—] and combinations thereof.

10. A method of producing a composition of matter having the molecular structure [I] 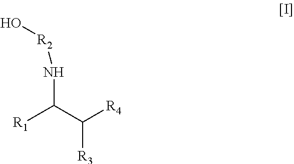
the method comprising;
(a) feeding an unsaturated organonitrile, a hydroxy-amine, and optionally, a solvent to a first reaction zone,
(b) maintaining the temperature and pressure in the first reaction zone for time sufficient to obtain a hydroxy-aminonitrile of the molecular structure [II]: 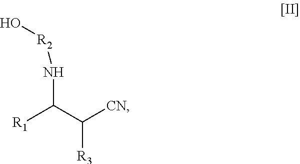
(c) feeding the hydroxy-aminonitrile obtained from step b) to a second reaction zone, and
(d) maintaining the reaction conditions in the second reaction zone for time sufficient for functionalizing the hydroxy-aminonitrile of the molecular structure [II] to obtain a composition of matter of the molecular structure [I]:
wherein,
R₁ is an alkyl, cycloalkyl, or aralkyl group having ≥1 to ≤10 carbon atoms which optionally comprises unsaturated fractions;
R₂—is selected from the group consisting of an aliphatic C₁-C₁₀ group, an unsaturated C₂-C₁₀ chain, a C₄-C₉ group with a cyclic portion, a C₄-C₁₀ group with an aromatic portion, and combinations thereof;
R₃ is selected from the group consisting of hydrogen, an aliphatic C₁-C₁₀ chain, an unsaturated C₂-C₁₀ chain, a C₄-C₁₀ group with a cyclic portion and a C₄-C₁₀ group with an aromatic portion, and combinations thereof, and
R₄ is a terminal functional group selected from the group consisting of amide [—CONH₂], acid [—COOH], amine [—CH₂—NH₂], guanamine [—(C₃N₃)—(NH₂)₂], organic heterocyclic [—CHN₄], and combinations thereof;

11. The method of claim 10, wherein the unsaturated organonitrile comprises a C₅ unsaturated nitrile.

12. The method of claim 11, wherein the C₅ unsaturated nitrile is selected from the group consisting of 2-pentenenitrile [2PN], 3-pentenenitrile [3PN], 4-pentenenitrile [4PN], 2-methyl-2-butenenitrile [2M2BN], 2-methyl-3-butenenitrile [2M3BN] and cis/trans mixtures thereof.

13. The method of claim 10, wherein the hydroxy-amine is selected from the group consisting of a C₁-C₁₀ alkanolamine, a C₂-C₁₀ unsaturated alkanolamine, a C₄-C₁₀ cyclic alkanolamine, a C₄-C₁₀ aromatic hydroxy-amine and mixtures thereof.

14. The method of claim 10, wherein the hydroxy-amine is selected from the group consisting of methanol amine, ethanol amine, butanol amine, pentanol amine, hexanol amine, heptanol amine, octanol amine, nonanol amine, decanol amine and isomers and mixtures thereof.

15. The method of claim 10, wherein the hydroxy-amine is selected from the group consisting of methanol amine, ethanol amine and mixtures thereof.

16. The method of claim 10, wherein the hydroxy-amine is selected from the group consisting of amino-cyclobutanol, amino-cyclopentanol, amino-cyclohexanol, amino-cycloheptanol, amino-cyclooctanol, amino-cyclononanol, amino-cyclodecanol and mixtures thereof.

17. The method of claim 10, wherein the hydroxy-amine is selected from the group consisting of 2-amino-cyclobutanol, 2-amino-cyclohexanol, 3-amino-cyclohexanol, 4-amino-cyclohexanol and mixtures thereof.

18. The method of claim 10, wherein the solvent is selected from the group consisting of water, alcohols, alkanes, ethers and esters.

19. The method of claim 10, wherein said functionalizing of the hydroxy-aminonitrile of the molecular structure [II] comprises a reaction selected from the group consisting of:
(i) hydrolytic reduction in the presence of water and a catalyst to obtain a hydroxy-amino-amide,
(ii) hydrolysis in the presence of water and a catalyst to obtain a hydroxy-amino-acid,
(iii) reductive hydrogenation in the presence of ammonia- and hydrogen-containing medium to obtain a hydroxy-amino-amine,
(iv) reaction with a dicyandiamide in the presence of a basic catalyst to obtain a hydroxy-amino-guanamine, and
(v) reaction with an azide salt and a catalyst presence to obtain a hydroxy-cyclic imine.
